(19) United States
(12) Patent Application Publication
(10) Pub. No.: US 2024/0058180 A1
(43) Pub. Date: Feb. 22, 2024

(54) ARRANGEMENT FOR DETECTING LIQUID

(71) Applicant: RKW SE, Mannheim (DE)

(72) Inventors: Robert KOPLIN, Dittenheim (DE); Florian BRANDMAIER, Koblermoor (DE); Michael SCHUHMANN, Cadlozburg (DE); Veronica SCHAFSTALLER, Muenchen (DE); Sarah JÄCKEL, Rosenheim (DE)

(73) Assignee: RKW SE, Mannheim (DE)

(21) Appl. No.: 18/027,408

(22) PCT Filed: Mar. 8, 2022
(86) PCT No.: PCT/EP2022/055914

§ 371 Date: Mar. 21, 2023

(30) Foreign Application Priority Data
Apr. 13, 2021 (DE) 10 2021 109 117.9

(51) Int. Cl. A61F 13/514; A61F 13/42

(52) U.S. Cl. CPC A61F 13/51401; A61F 13/42; A61F 2013/424

(57) ABSTRACT

An arrangement (4) for detecting liquid in a single-use hygiene article. The arrangement (4) includes a back sheet film (1) with a conductive print (2).

### CROSS-REFERENCE TO RELATED APPLICATIONS

This application is a 371 National Phase of International Application No. PCT/2022/055914, filed Mar. 8, 2022, which claims priority from German Patent Application No. 10 2021 109 117.9, filed Apr. 13, 2021, both of which are incorporated by reference herein as if fully set forth.

### TECHNICAL FIELD

The invention relates to an arrangement for detecting fluids in a single-use hygiene article, and to a process for producing such articles and the use thereof.

### BACKGROUND

Single-use hygiene articles for babies and adults are customary in particular in the form of diapers. Conventional single-use hygiene articles of this kind possess a sheetlike, longitudinally extended absorbent element which when the single-use hygiene article is donned passes through the crotch of the wearer. The absorbent element frequently comprises superabsorbent material.

The absorbent element is disposed on an arrangement which comprises a polyolefinic backsheet film. The arrangement is to give the sensation of a textile. For this purpose, preferably, the backsheet film is joined to a nonwoven ply. The arrangement carries the absorbent element and seals it off from the outside.

There is increasing demand for products, under the heading “intelligent” single-use hygiene articles, which are capable of detecting their usage status. Babies or dementia patients are unable themselves to ascertain the status of the single-use hygiene article and/or to let others know said status. Monitoring by human auxiliaries is complicated.

A solution is provided by intelligent single-use hygiene articles which automatically indicate when a change is needed. This increases the quality of life and relieves the burden on the care staff. An intelligent diaper also makes a contribution to environmental protection, since the potential of the single-use articles is fully utilized before disposal.

Against this background, a variety of approaches are known from the prior art for intelligent single-use hygiene articles.

U.S. Pat. No. 6,200,250 B1 relates to an intelligent diaper wherein capacitive electrodes are sited on a carrier film and on a fluid-absorbing arrangement. The electrodes here may be rectangular in design and be connected in series or configured as filaments. The electrodes here may be secured in the diaper by adhesive bonding or stitching.

According to U.S. Pat. No. 8,978,452 B2 and also to WO 2013/022742 A1, fluid sensors with a passive resonant circuit are proposed for a film portion, where the structures applied can be partly undone by a fluid.

In U.S. Pat. No. 8,978,452 B2, the status of a hygiene article, detected by a reading device, may be subsequently transmitted, including wirelessly, to further devices by way of known data communication links such as WLAN and Bluetooth.

DE 10 2017 125 323 A1 describes a film portion having two conductive tracks which form a passive resonant circuit. Connected to the conductor tracks is a reading and transmitting device. One conductor track is configured as a capacitor plate, and the other conductor track as a coil. These conductor tracks are applied on a carrier film which is in turn disposed on a film label. The film label is enclosed by a PE film and adhered on the rear wall of a hygiene article. This film label must be mounted on the rear wall of the hygiene article in an additional working step, with a disproportionate increase in the volume of the hygiene article. The additional volume may adversely affect the wear sensation of the hygiene article. Furthermore, the film label itself is improbably complex in its construction and also to realize in production.

EP 2 654 646 B1 describes an absorbent article which comprises a conductive and an open conductor loop. A fluid absorbed by an absorbent core measurably changes the impedance of the conductor loops. This operates by the conductor loops being aligned such that the current generates a short-circuit on an outer leg of the second conductor loop.

EP 3 451 988 B1 and EP 3 760 104 A1 describe a fluid-impervious backing with a nonconducting insulating layer on which at least one channel for a sensor track is disposed. Preferably, in a colored reinforcing layer in the form of a sew-on patch, sensor tracks are disposed which with a data processing module form a left-hand and a right-hand current circuit for the recognition of moisture events.

According to EP 3 143 974 B1, a transmitter-receiver arrangement for monitoring the absorption layer may be coupled detachably to single-use hygiene articles. This arrangement can then be utilized more than once or multiply on single-use hygiene articles. This allows the focus of future developments to be placed on the provision of a favorable single-use hygiene article.

Electrodes and conductor tracks are complicated and expensive. Single-use hygiene articles are subject to price pressure and to exacting environmental requirements, as they are disposed of directly after their one-off usage. Furthermore, there is also a desire for maximum ease of handling.

The inventions described consist mostly of multiple layers having different properties and requiring joining to one another. The resulting single-use hygiene articles are thick and heavy. This promotes the formation of pressure points and skin irritation among those wearing the diapers, resulting in an uncomfortable wear sensation. Moreover, intelligent single-use hygiene articles ought not to give anything away to their conventional counterparts in terms of their tactile qualities.

### SUMMARY

It is an object of the invention to provide a simple and compact arrangement for detecting fluids. The arrangement is to be fluid-impervious, to meet the required mechanical properties and to ensure a pleasing wear comfort. The arrangement is also to have tactile appeal. An objective is to provide an arrangement which causes very little rustling noise and which possesses elasticity and softness. The arrangement is also to provide conductive elements which are necessary for reliable detection of fluids and can nevertheless be produced inexpensively. The arrangement is to heighten the quality of the single-use hygiene article and to satisfy the requirements for producing it in modern operations.

This object is achieved in accordance with the invention by an arrangement for detecting fluids, and by a process and a use. Preferred variants are apparent from the description below, claims, and the examples.

In the invention the arrangement for detecting fluids comprises a backsheet film having a conductive print which has an area-specific resistance or sheet resistivity of less than 10 kM. In a particularly advantageous variant of the invention the area-specific resistance of the conductive print is less than 6 kM, preferably less than 4 kM, more particularly less than 1 kM.

In one advantageous variant of the invention the surface weight of the conductive print is less than 3 g/m², preferably less than 2/m², more preferably less than 1 g/m², more particularly less than 0.5 g/m². Where, for example, there is a wet application of 2*3.5 g of conductive medium per m² with a coverage of 11% and a conductive medium solids fraction of 35%, the result is a surface weight of the dry conductive print of 7 g/m²*0.11*0.35=0.2695 g/m².

The area-specific resistance is a product of the amount of a conductive printing ink applied, the thickness of the print, and a type of treatment. The area-specific resistance has its lowest value immediately after print application. An alteration of this area-specific resistance is critically dependent on the properties of the backsheet film, more particularly on the combination of stiffness and elasticity. Only as a result of the backsheet film of the invention, which has a particularly stiff and at the same time elastic configuration, does the print-applied, area-specific resistance remain extremely low during further processing as well. The use of specific, polyolefinic backsheet films ensures that the conductor tracks of the print are not damaged, by ruptures, for example, during the further processing steps.

The area-specific resistance of the print may be captured using a multimeter via two measurement peaks.

The area-specific resistance of the print may be captured by means of the four-point method or in the form of a contactless measurement using a special-purpose eddy current tester. It is often a result of a back-calculation based on a known geometry. In the case of the four-point method, the effect of the contact resistance on the measurement is eliminated by generating a current flow between two contact points, while measuring the drop in current over two further contact points. In the case of the contact-free measurement of the film resistance using eddy current, an alternating electromagnetic field is generated in the material, the opposing field to which is evaluated by the measuring sensor.

The sheet resistivity R_{□} describes the electrical resistance of an electrically conductive layer whose thickness is such that electrical current flows through it only parallel to the layer, meaning that the current enters at one end face and leaves again at the opposite end face.

The area specific resistance R_{□} of a backsheet film is measured preferably at a printed-on strip of the conductive print. The strip, for example, has a length of 30 cm and a width of 6 mm, corresponding in this case to 50_{□}, which in the case of a measured resistance of 60 kΩ, for example, would produce an area-specific resistance of 1.2 kΩ/□.

For more effective differentiation from the electrical resistance with the unit 0, therefore, the sheet resistivity R_{□} is often expressed in the unit Ω/□. However, the standards Din 1301 and ISO 31 make no provision for such indication of physical units, and so the claims give the unit 0 for the sheet resistivity R_{□}.

The print may be implemented as a printed pattern particularly suitable for fluid detection. In the simplest case the printed pattern is a strip. Alternatively it may be configured in the form of multiple strips which extend over the arrangement. Furthermore, the printed motif may also be implemented spirally, triangularly, rectangularly and/or in a completely random geometric formation. In one variant the conductor tracks have a checkerboard configuration.

The printing inks to be used for the conductive print are preferably printing inks having a low viscosity and are therefore of virtually water consistency. Suitable in principle are aqueous, UV-curable, and solvent borne ink systems. These printing inks may be adapted specifically to the respective printing process and are therefore adapted individually to the particular printing machine.

Conductive printing inks may be dried and/or cured in particular through heat/and or UV light. Conductive printing inks generally include particle of conductive materials, such as graphite, copper or silver, for example, and the particles may be provided in flake or powder form.

In the context of the invention, preferably, a conductive printing ink based on graphite is preferred. The conductive print is implemented preferably on the basis of a carbon-based ink and/or of a conducting, polymer-based ink. The carbon-based ink preferably comprises a conducting compound which is formed from the group consisting of graphene, graphite, carbon nanotubes, and mixtures thereof. A conducting polymer-based ink preferably comprises a conducting compound which consists of the group of polyacetylene, polypyrrole, polyaniline, and copolymers thereof. The conductive ink may more particularly consist of polypyrroles (PPY), polyanilines (PANI), polythiophenes (PT), polyphenylene sulfide (PPS), polyphenylene (PPP), polyacetylenes (PAC), polyphenylene-vinylene (PPV), poly(3,4-ethylenedioxythiophene) (PEDOT), and mixtures thereof, with the conducting, polymer-based ink ideally comprising a poly(3,4-ethylenedioxythiophene) polystyrene sulfonate (PEDOT:PSS).

The conductive print is applied preferably with a flexographic printing process, with all common printing processes being suitable in principle for this purpose and being expressly included in the invention.

The conductive print on the backsheet film advantageously exhibits no abrasion by the ink rub-off test. As a result, irrespective of the arrangement of the print during the wearing of the hygiene article, there is no possibility for transfer of the print to the skin of the wearer of the hygiene article.

In the ink rub-off test, a test arm with fabric-like material is placed onto the printed backsheet film. The platform then moves back and forth beneath the backsheet film 15 times. This is followed by evaluation of the transfer of ink to the fabric-like material, where pass without ink abrasion, pass with slight ink abrasion, and fail represent the three evaluation categories.

The arrangement in the invention comprises a specific combination of features which are unknown in conventional arrangements for fluid detection according to the prior art. As a result of this specific combination of features it is possible to use even thin and particularly simple arrangements which have a conductive print.

Relative to conventional arrangements for intelligent diapers, the arrangement in the invention has much better mechanical properties, is particularly soft and gentle, and does not employ any complicated electrical connections or complex constructions thereof at all. The arrangement in the invention is free from wires, electrical conductor tracks, resistance circuits, and the associated additional insulating layers.

The arrangement in the invention is particularly thin and at the same time stiff, and so the print does not suffer any damage during processing in modern diaper converters. Nevertheless the arrangement in the invention provides reliable assurance against drenching, in spite of its simple construction from a backsheet film, a conductive print, and a nonwoven.

In one variant of the invention the arrangement comprises a nonwoven ply. The expression “nonwoven”, refers to a material which can be produced from continuous filaments and/or discontinuous fibers without weaving or knitting, by means of processes such as spun bonding, carding or melt-blowing. The nonwoven fabric may comprise one or more plies of nonwoven, and each ply may comprise continuous filaments or discontinuous fibers. Nonwoven may also comprise bicomponent fibers, which may have fiber structures such as, for example, core/shell or side-by-side.

The nonwoven ply preferably consists of a polyolefinic nonwoven, more particularly a thermally bonded spun web. Especially suitable in this context is a polypropylene-based nonwoven fabric. The nonwoven ply preferably has a specific weight of more than 6 g/m², more preferably of more than 8 g/m², more particularly of more than 10 g/m² and/or less than 20 g/m², more preferably less than 18 g/m², more particularly less than 16 g/m².

Disposed between the nonwoven ply and the backsheet film are joining regions. The joining regions are configured advantageously as a form-fitting composite of nonwoven and solidified material of the backsheet film. This is achieved by heating the thermoplastic polymer material of a backsheet film beyond the crystallite melting point of the polymer material, in a process for producing an arrangement composed of a backsheet film and a nonwoven ply, and subsequently passing the heat-treated backsheet film together with the nonwoven ply through a cooled roll nip.

In one particularly advantageous variant of the invention the conductive print is disposed on that side of the backsheet film on which the nonwoven ply is joined by thermal lamination. This is only possible because of the particular stiffness of the backsheet film and because the conductive print as a result cannot rupture during further processing.

It proves particularly favorable if the print is disposed directly on the backsheet film. Accordingly it is possible, for example, to do without the application of an adhesion promoter, so saving on an additional operating step. At the same time, the direct application of the print allows the arrangement to be made particularly thin. Nor is there any need, moreover, to introduce additional elements, for the detection of a fluid, for example. This is especially advantageous since there are no potential sources of error arising from the mounting of additional elements and from the performance of further working steps such as the unwinding of the film web and the mounting of the elements.

The arrangement is produced preferably in a process in which a specific composition is first extruded to form a film web, preferably by means of blown film extrusion, and is cooled for further processing. The film web is then oriented in machine direction and/or transversely to the machine direction. In the invention the conductive print is applied in an in-line operation. In the case of the in-line operation, the film web does not have to be wound up and collected up again before being joined with a nonwoven ply. The nonwoven ply is applied “in-line” and therefore preferably immediately after the application of the conductive print, in one operation. To conclude the operation, the nonwoven can be mobilized. This is accomplished preferably by means of ring rolling. The softness impression of the arrangement is boosted as a result.

The print is disposed ideally between the backsheet film and the nonwoven ply. The in-line process of the invention enables a form-fitting composite to be formed between backsheet film and nonwoven, without adversely affecting the conductivity of the print.

The process of the invention is particularly advantageous because the completed arrangement can be rolled up in webs without the film web having to be rolled up separately following the application of the conductive print, which could result in this print being damaged by further exposure to stress and its conductivity being reduced.

The backsheet film preferably comprises thermoplastic fractions, and it proves particularly favorable if the polymer materials of backsheet film and nonwoven ply are harmonized with one another so that firstly the crystallite melting points are sufficiently far apart and secondly the materials are compatible with one another to an extent which allows them to be joined.

The difference in the crystallite melting temperature of the low-melting component of the backsheet film ought to be at least about 5° C., preferably at least about 10° C., and more particularly at least about 20° C. below the melting temperature of the nonwoven or below the melting temperature of the high-melting component of the nonwoven.

In the invention the backsheet film preferably comprises at least one low-melting polymer component and at least one high-melting polymer component.

The total amount of low-melting polymer component is preferably 90 to 30 wt %, more particularly 80 to 40 wt %, most preferably 70 to 50 wt %, and the total amount of high-melting polymer component is preferably 10 to 70 wt %, more particularly 20 to 60 wt %, most preferably 30 to 50 wt %, based in each case on 100 wt % of low-melting and high-melting polymer components.

In one embodiment the backsheet film comprises at least one polyethylene as low-melting polymer component and at least one polypropylene as high-melting polymer component.

In the case of a variant, the low-melting polymer component comprises or consists of ethylene polymers, with not only ethylene homopolymers but also ethylene copolymers with ethylene as principal monomer and also blends of ethylene homopolymers and ethylene copolymers being suitable. Suitable ethylene homopolymers are LDPE (low density polyethylenes), LLDPE (linear low density polyethylenes), MDPE (medium density polyethylenes) and HDPE (high-density polyethylenes).

In one embodiment the low-melting polymer component consists exclusively of ethylene homopolymers, e.g., of mixtures of LDPE and LLDPE, which may each be present in amounts of 10 to 90 wt %, and also of 0 to 50 wt % of MDPE.

The high-melting polymer component preferably comprises at least one polypropylene whose melting point, melting range or crystallite melting point is substantially higher than that of the low-melting polymer component. Especially suitable polypropylene is isotactic polypropylene. Syndiotactic polypropylene can also be used, provided its melting point, melting range or crystallite melting point is substantially higher than that of the low-melting polymer component.

The high-melting polymer component may comprise not only propylene homopolymers but also propylene copolymers with propylene as principal monomer.

The backsheet film webs and nonwoven plies to be joined preferably have a similar morphology at least in one formula component.

In the invention the backsheet film is heated together with the nonwoven ply using a preferably nonstick-coated heating cylinder and is subsequently passed through a cooled roll nip. It is of course also possible to operate with multiple heating cylinders or with other heating methods such as infrared lamps, for example.

In one preferred embodiment the nonwoven ply is in direct contact with the heating cylinder surface. The backsheet film is passed over this surface together with the conductive print. The temperature of the heating cylinder is selected such that, over the film contact section of the heating cylinder, a component of the backsheet film is heated to the liquid-melt state without impairing the conductive print. This temperature is not enough to render the nonwoven into the liquid-melt state.

Since the nonwoven not yet in the liquid-melt state is lying on the heating cylinder, it is easy to detach the back sheet film, and this operation is very stable.

In the downstream cooled rolled nip, the nonwoven-film composite is cooled to temperatures below the crystallite melting point of the backsheet film. The cooled roll nip consists preferably of a steel roll and a rubber roll which operates with opposing pressure.

In contrast to known thermal-bonding lamination processes, in which the composite is produced only locally by means of two heated steel rollers, applying temperature and very high pressures, the thermal lamination process generates joining regions featuring lamination over the full area. This offers the advantage, similarly to the known adhesive lamination processes, that very elastic arrangements can be created without adhesive by virtue of low pressures in the laminating operation. Furthermore, in contrast to thermal-bonding laminates, there is no risk of damages to the material (apertures, pin holes) which is extremely important particularly with regard to the conductive print.

In an alternative variant of the invention, it is conceivable for the arrangement composed of nonwoven ply and backsheet film to be joined via a hotmelt adhesive. Joining of the film web to a nonwoven ply by way of an ultrasound welding procedure is also conceivable.

The film may be printed after the extrusion and before the lamination. This allows printing to take place onto the backsheet film side which in the subsequent arrangement is covered by the nonwoven ply. The result is a very good print quality, since printing can take place onto the smooth backsheet film. As a result, the conductive print can be employed for fluid detection and at the same time the backsheet film provides fluid-impervious sealing of the arrangement. The joined nonwoven ply also protects the conductive print in the further processing to form the single-use hygiene article.

The printing is preferably integrated in-line into the production operation. Following the extrusion of the backsheet film there is preferably an embossing mechanism and/or a chili-roll system and/or at least one chill roll.

In the case of one variant, the nonwoven-film laminate is subjected to ring rolling. This gentle operating step mobilizes the fibers and increases the elasticity and the softness of the laminate. These property alterations described can be easily influenced by the geometry used and by the degree of engagement of the ring-rolling rolls. Only the properties of the backsheet film provided by the invention enable gentle treatment by ring rolling, without critically altering the area-specific resistance of the conductive print.

In the case of one variant of the invention, the backsheet film has a non-breathable configuration and an ASTM D6701-01 water vapor transmission rate of less than 500 g/m² in 24 h. The surface weight of the non-breathable backsheet film is preferably less than 10 g/m², preferably less than 8 g/m², more particularly less than 6 g/m².

The ASTM D1709A specific dart drop of the arrangement in this case is preferably more than 6 g per gram of polymer per square meter, and so the value is obtained from the mass of the “dart” divided by the specific weight of the film-nonwoven laminate and therefore 6 g/(g/m²), and/or the specific water column of the arrangement is more than 270 mm per gram of polymer per square meter, and so the value is obtained from the water column divided by the specific weight of the film-nonwoven laminate and is therefore 270 mm/(g/m²).

These advantageous properties of the arrangement according to the invention are manifested in characteristic values. The arrangement has a force of at least 0.3 N/in, preferably a force of at least 0.375 N/in, more particularly a force of more than 0.45 N/, in each case at 5% elongation, per gram of polymer per square meter, and so the value is obtained from the force per inch divided by the specific weight of the film-nonwoven laminate.

If, for example, a value of 8.5 N/in is measured, for a film-nonwoven laminate having a specific weight of 18 g/m², the resulting value is 0.47222 (N/in)/(g/m²).

The force at 5% elongation is measured according to ASTM D882. As a result, the non-breathable backsheet film is particularly stiff, thus providing particular protection from print rupture to a conductive print. Because of the formula according to the invention, moreover, the backsheet film is very elastic, which contributes advantageously to the protection of the conductive print and to the generation of a pleasing tactility.

In a further variant of the invention, the backsheet film has a breathable configuration. The breathable backsheet film has an ASTM D6701-01 water vapor transmission rate of more than 500 g/m² in 24 h. The surface weight of the breathable backsheet film is preferably less than 20 g/m², more preferably less than 16 g/m², more particularly less than 12 g/m².

The ASTM D1709A specific dart drop of the breathable backsheet film is preferably more than 26 g per gram of polymer per square meter and/or the specific water column of the breathable backsheet film is more than 460 mm per gram of polymer per square meter.

The particular mechanical properties of the breathable backsheet film have their basis in a specific formula in combination with a very specific processing, with preferably blown film extrusion being employed in the production of the film.

In the case of one variant of the invention, the backsheet film comprises a low-melting polypropylene and a high-melting polypropylene.

In this case it proves particularly if the difference in the crystallite melting point between the low-melting polypropylene and the high-melting polypropylene in the backsheet film is more than 10°, preferably more than 15° C., more particularly more than 20° C. and/or less than 50° C., preferably less than 40° C., more particularly less than 30° C.

In the case of one variant of the invention, the composition preferably comprises CaCO₃ with a fraction of 40 to 60 wt %. In a particularly advantageous variant of the invention, the composition of the breathable backsheet film comprises an LDPE with a fraction of more than 2 wt % and/or less than 10 wt %.

The breathable backsheet film preferably exhibits a force of at least 0.5 N/in, more preferably a force of more than 0.6 Nfin, more particularly a force of more than 0.7 N/in per gram of polymer per m², in each case at 5% elongation. The force at 5% elongation is measured according to ASTM D882. The breathable backsheet film is very stiff, and so the conductive print is protected from possible damage. Furthermore, the backsheet film is highly elastic, so producing a pleasing tactility.

If, for example, a value of 4.2 N/in at 5% elongation is measured, for a film having a specific weight of 16 g/m² and a polymer fraction of 40%, the resulting value is (4.2 N/in/16 g/m²)/0.4=0.65625 (N/in)/(g/m²).

The arrangement according to the invention therefore has a particular lightness, so enabling a pleasing wear sensation to be generated.

This favorable stiffness in combination with an advantageous softness of the arrangement is achieved through a specific composition, a targeted selection of polymers in combination with a specific production operation.

The arrangement according to the invention for fluid detection is used preferably in the hygiene or medical sector, especially for single-use hygiene articles. These single-use hygiene articles may be used by both the youngest and the oldest in our society.

From the single-use hygiene article, the area-specific resistance can be read out with a reading device, which can be clipped onto the single-use hygiene article and which forms a detection unit with the conductive printed pattern. As a result, the single-use hygiene article becomes an intelligent diaper. If an adjacent fluid is in conducting communication with the conductive printed pattern, a distinct change in resistance can be ascertained with the reading device. The reading device preferably has a Bluetooth transmitting/receiving unit, allowing a moisture event to be transmitted and displayed independently of spatial proximity.

### Example 1: Arrangement for detecting liquids, having a breathable backsheet film

In this example, the following components are employed for producing the backsheet film:

| | | Density, | Crystallite melting |
|---|---|---|---|
| Amount, wt % | Constituent | g/cm³ | point, ° C. |
| 32 | PP | 0.9 | 140-142 |
| 58 | CaCO₃ | — | |
| 6 | PP | 0.9 | 163-167 |
| 4 | LDPE | 0.92 | 111 |

The filler employed is an inorganic filler in the form of calcium carbonate, preferably with a particle size of 0.8 to 2 μm.

To produce the backsheet film of the invention, the polymer constituents with the mineral fillers are heated to a temperature well above the melting temperature of the polymer constituents (for example, above 200° C.) and fused with another in an extruder, such as a compounding extruder, for example.

This is followed, in the invention, by blown film extrusion with cooling of the film web. In the subsequent monoaxial orientation procedure, the backsheet film is drawn preferably 100% in machine direction.

In the in-line process of the invention, printing with conductive printing ink by a flexographic printing process takes place directly onto the breathable backsheet film. The conductive printing ink comprises preferably graphite pigments, which are in solution in a mixture of propyl acetate and propan-2-ol. The conductive, black printing ink preferably comprises fillers such as cellulose nitrate.

The backsheet film is then guided with a polypropylene-based, thermally bonded nonwoven ply having a specific surface weight of 14 g/m² over a heating cylinder surface, so that the backsheet film is partly in the melted state and forms extensive joining regions with the nonwoven ply in the downstream cooled roll nip. The conductive print on the breathable backsheet film is covered by the joined nonwoven ply. In a final ring rolling, the fibers of the film-nonwoven composite are gently activated, producing an appealing soft tactility.

The area-specific resistance of the conductive print is determined from a test strip having a length of 30 cm and a width of 6 mm, corresponding to 50 squares. In this procedure, the measuring tips of a multimeter are pressed on at the ends and a resistance value of, for example, 57 kΩ is measured. By computation this gives an area-specific resistance of 1.14 kΩ/squares.

### Example 2: Arrangement for detecting liquids, having a non-breathable backsheet film

In this example, the following components are employed:

| | | Density, | Crystallite melting |
|---|---|---|---|
| Amount, wt % | Constituent | g/cm³ | point, ° C. |
| 37.5 | LDPE | 0.92 | 111 |
| 25.5 | LLDPE | 0.92 | 123 |
| 16.25 | PP | 0.90 | 163-167 |
| 7.5 | PP | 0.90 | 140-142 |
| 8.0 | TiO₂ | — | |
| 4.5 | CaCO₃ | — | |
| 0.5 | Heat stabilizer | 0.93 | |
| 0.25 | Auxiliary | 0.94 | |

To produce the film web of the invention, the polymer constituents are heated to a temperature well above the melting temperature of the polymer constituents, and fused with another, in an extruder, such as a compounding extruder, for example. This is followed by blown film extrusion, with cooling of the multilayer film web. In the in-line process of the invention, the print is applied with conductive printing ink by a flexographic printing process directly to the non-breathable backsheet film. An alternative conductive printing ink comprises typically graphite pigments, which are in solution in a mixture of water and ammonium hydroxide.

The non-breathable backsheet film is then guided with a polypropylene-based, thermally bonded nonwoven ply having a specific surface weight of 14 g/m² over a heating cylinder surface, so that the backsheet film is partly in the melted state and forms extensive joining regions with the nonwoven ply in the downstream cooled roll nip. In a final ring rolling, the fibers of the film-nonwoven composite are gently activated, producing an appealing soft tactility.

The area-specific resistance of the conductive print is determined from a test strip having a length of 30 cm and a width of 6 mm, corresponding to 50_{□}. In this procedure, the measuring tips of a multimeter are pressed on at the ends and a resistance value of, for example, 56 kM is measured. By computation this gives an area-specific resistance of 1.12 kΩ/□.

### BRIEF DESCRIPTION OF THE DRAWINGS

Further advantages and features of the invention are apparent from the description of various exemplary embodiments with reference to drawings, and from the drawings themselves.

In these drawings

FIG. 1 shows a schematic representation of a backsheet film with a print,

FIG. 2 shows a schematic representation of the arrangement according to the invention,

FIG. 3 shows a further variant of the arrangement according to the invention, and

FIG. 4 shows a third variant of the arrangement according to the invention.

### DETAILED DESCRIPTION

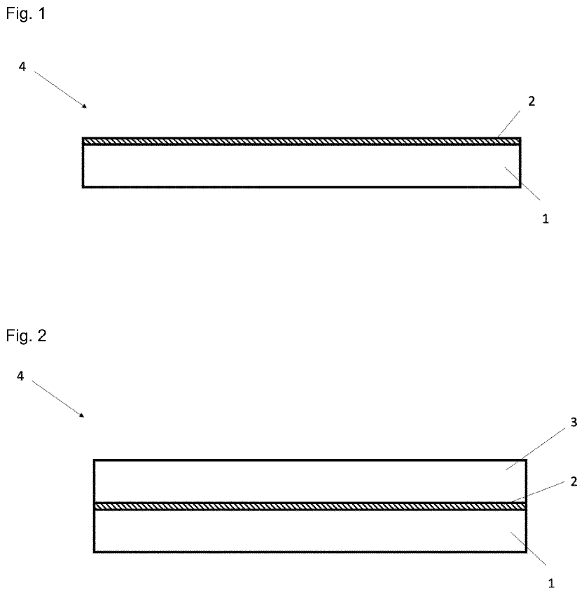

Represented in FIG. 1 is a backsheet film 1 having a conductive print 2, the conductive print 2 being disposed directly on the backsheet film 1 without an adhesion promoter layer. The backsheet film 1 is breathable in implementation and has a specific surface weight of 12 m²/g. The surface weight of the conductive print is less than 0.5 g/m².

FIG. 2 shows an arrangement according to FIG. 1, where additionally a nonwoven ply 3 is joined to the backsheet film 1. The nonwoven ply 3 has a specific surface weight of 12 g/m².

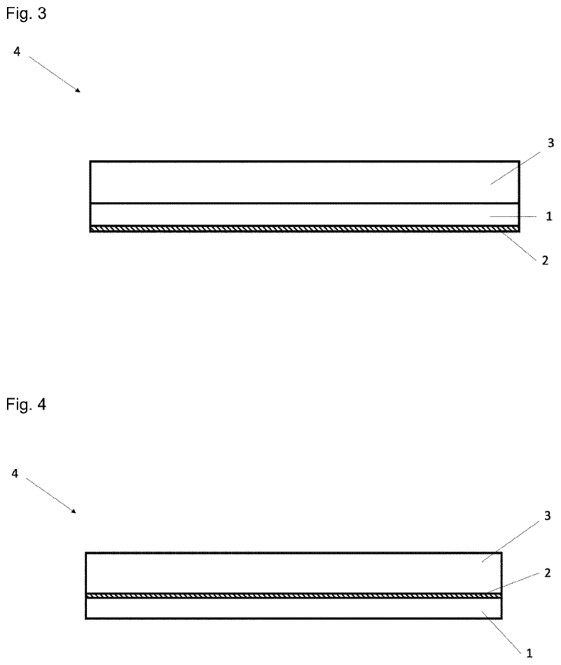

Represented in FIG. 3 is an arrangement wherein the backsheet film 1 is provided with a conductive print 2, with the nonwoven ply 3 being disposed on the opposite side of the conductive print 2.

FIG. 4 shows a version according to FIG. 2, in which the backsheet film 1 is not breathable in implementation and has a surface weight of 6 g/m².

1. An arrangement for detecting fluid in a single-use hygiene article, the arrangement comprises a backsheet film with a conductive print thereon.

2. The arrangement as claimed in claim 1, wherein the conductive print has an area-specific resistance of less than 10 kΩ.

3. The arrangement as claimed in claim 1, wherein a surface weight of the conductive print is less than 3 g/m².

4. The arrangement as claimed in claim 1, wherein the backsheet film printed with the conductive print exhibits no abrasion by a rub-off test.

5. (The arrangement as claimed in claim 1, further comprising a nonwoven ply with joining regions disposed between the nonwoven ply and the backsheet film.

6. The arrangement as claimed in claim 5, wherein the joining regions are configured as a form-fitting composite of material of the nonwoven ply and solidified material of the backsheet film.

7. The arrangement as claimed in claim 5, wherein the conductive print is disposed between the backsheet film and the nonwoven ply.

8. The arrangement as claimed in claim 1, wherein the backsheet film has a non-breathable configuration and an ASTM D6701-01 water vapor transmission rate of less than 500 g/m² in 24 h, with a surface weight of the backsheet film being less than 10 g/m².

9. The arrangement as claimed in claim 8, wherein at least one of
a specific dart drop of the arrangement is more than 6 g per gram of polymer per square meter or
a specific water column of the arrangement is more than 270 mm per gram of polymer per square meter.

10. The arrangement as claimed in claim 8, wherein the arrangement has
a force of at least 0.3 N/in per gram of polymer per m².

11. The arrangement as claimed in claim 1, wherein the backsheet film has a breathable configuration and an ASTM D6701-01 water vapor transmission rate of more than 500 g/m² in 24 h, with a surface weight of the backsheet film being less than 20 g/m².

12. The arrangement as claimed in claim 11, wherein at least one of
a specific dart drop of the backsheet film is more than 26 g per gram of polymer per square meter or
a specific water column of the backsheet film is more than 460 mm per gram of polymer per square meter.

13. The arrangement as claimed in claim 11 the backsheet film has a force of
at least 0.5 N/in per gram of polymer per m².

14. The arrangement as claimed in claim 1, wherein the conductive print is disposed directly on the backsheet film.

15. A process for producing an arrangement for detecting fluid, the process comprising:
producing a composition,
extruding the composition to form a film web,
cooling the film web,
applying a conductive print to the film web, and
joining the film web to a nonwoven ply.

16. The process as claimed in claim 15, wherein the film web is oriented.

17. The process as claimed in claim 15, wherein the film web is embossed.

18. The process as claimed in claim 15, wherein the conductive print is applied between the film web and the nonwoven ply.

19. The process as claimed in claim 15, wherein the film ply is joined to the nonwoven ply in-line in an operation.

20. The process as claimed in claim 15, further comprising joining the film web in a partly melted state to the nonwoven ply in a cooled roll nip.

21. The process as claimed in claim 15, further comprising subjecting the arrangement to a ring rolling operation.

22. A process for detecting fluid, the process comprising using the arrangement of claim 1 in single-use hygiene articles.
